(19) United States
(12) Patent Application Publication
(10) Pub. No.: US 2025/0139526 A1
(43) Pub. Date: May 1, 2025

(54) METHOD FOR TRAINING AN ARTIFICIAL INTELLIGENCE MODEL AND ELECTRONIC APPARATUS THEREFOR

(71) Applicant: Smartsound Corporation, Seoul (KR)

(72) Inventors: Jungho LEE, Seoul (KR); Jae Yong KIM, Seoul (KR); Won Yang CHO, Yongin-si (KR); Hye Sun CHANG, Seoul (KR)

(21) Appl. No.: 18/929,422

(22) Filed: Oct. 28, 2024

(30) Foreign Application Priority Data
Oct. 30, 2023 (KR) 10-2023-0146618

(51) Int. Cl. G06N 20/00; A61B 5/00; A61B 7/04

(52) U.S. Cl. CPC G06N 20/00; A61B 5/7267; A61B 7/04

(57) ABSTRACT

Provided is an artificial intelligence model training method of an electronic apparatus, the artificial intelligence model training method including primarily updating a second artificial intelligence based on first auscultation sound data and output data of a first artificial intelligence model, updating the first artificial intelligence model based on second auscultation sound data, bioacoustics data with noise removed from the second auscultation sound data, output data of the second artificial intelligence model, and output data of the primarily updated second artificial intelligence model, secondarily updating the primarily updated second artificial intelligence model based on third auscultation sound data and output data of the updated first artificial intelligence model, and tertiarily updating the secondarily updated second artificial intelligence model based on a reward corresponding to output data of the secondarily updated second artificial intelligence model for fourth auscultation sound data.

### CROSS-REFERENCE TO RELATED APPLICATION(S)

This application claims the benefit of Korean Patent Application No. 10-2023-0146618, filed on Oct. 30, 2023, in the Korean Intellectual Property Office, the disclosure of which is incorporated herein by reference.

### BACKGROUND

### Technical Field

The present disclosure relates to an electronic apparatus for training an artificial intelligence model that outputs enhanced auscultation sound data based on auscultation sound data and a control method thereof.

### Description of the Related Art

As use of the Internet become common and life span of population is increased, an interest in a home healthcare market is currently increased. Home healthcare is a service with which a user remotely receives medical care, treatment, and support without visiting a hospital.

Particularly, a service in which an auscultation sound of a body region such as a heart, a lung, an intestine is measured with a smart stethoscope and information on whether a disease is present is provided or the disease is diagnosed is spotlighted. Accordingly, in order to further accurately diagnosing a disease, a method for effectively removing noise from auscultation sound data is required.

### BRIEF SUMMARY

An aspect provides an electronic apparatus and an information management method thereof. More specifically, an aspect provides an electronic apparatus for training an artificial intelligence model that outputs enhanced auscultation sound data based on auscultation sound data and a control method thereof.

However, the goals to be achieved by example embodiments of the present disclosure are not limited to the objectives described above and other objects may be clearly understood from the following example embodiments.

According to an aspect, there is provided an artificial intelligence model training method of an electronic apparatus, the artificial intelligence model training method including primarily updating a second artificial intelligence based on first auscultation sound data and output data of a first artificial intelligence model, updating the first artificial intelligence model based on second auscultation sound data, bioacoustics data with noise removed from the second auscultation sound data, output data of the second artificial intelligence model, and output data of the primarily updated second artificial intelligence model, secondarily updating the primarily updated second artificial intelligence model based on third auscultation sound data and output data of the updated first artificial intelligence model, and tertiarily updating the secondarily updated second artificial intelligence model based on a reward corresponding to output data of the secondarily updated second artificial intelligence model for fourth auscultation sound data.

The primarily updating of the second artificial intelligence model may include acquiring first enhanced auscultation sound data by inputting the first auscultation sound data to the first artificial intelligence model with acquiring second enhanced auscultation sound data by inputting the first auscultation sound data to the second artificial intelligence, acquiring a first loss between the first enhanced auscultation sound data and the second enhanced auscultation sound data, and updating the second artificial intelligence model so that the first loss is minimized.

The updating of the first artificial intelligence model may include acquiring third enhanced auscultation sound data by inputting the second auscultation sound data to the first artificial intelligence model with acquiring fourth enhanced auscultation sound data by inputting the second auscultation sound data to the second artificial intelligence model and acquiring fifth enhanced auscultation sound data by inputting the second auscultation sound data to the primarily updated second artificial intelligence model, acquiring a second loss between the third enhanced auscultation sound data and the bioacoustics data, a third loss between the fourth enhanced auscultation sound data and the bioacoustics data, and a fourth loss between the fifth enhanced auscultation sound data and the bioacoustics data, and updating the first artificial intelligence model so that a sum of the second loss, the third loss, and the fourth loss is minimized.

The secondarily updating of the primarily updated second artificial intelligence model may include acquiring sixth enhanced auscultation sound data by inputting the third auscultation sound data to the updated first artificial intelligence model with acquiring seventh enhanced auscultation sound data by inputting the third auscultation sound data to the primarily updated second artificial intelligence model, acquiring a fifth loss between the sixth enhanced auscultation sound data and the seventh enhanced auscultation sound data, and updating the primarily updated second artificial intelligence model so that the fifth loss is minimized.

The first artificial intelligence model may include an artificial intelligence model trained in advance based on fifth auscultation sound data and bioacoustic data with noise removed from the fifth auscultation sound data.

The tertiarily updating of the secondarily updated second artificial intelligence model may include acquiring eighth enhanced auscultation sound data by inputting the fourth auscultation sound data to the secondarily updated second artificial intelligence model, acquiring the reward which corresponds to the eighth enhanced auscultation sound data by inputting the fourth auscultation sound data and the eighth enhanced auscultation sound data to a reward model, and updating the secondarily updated second artificial intelligence model so that the reward is maximized.

The reward model may include an artificial intelligence model supervised-learned based on sixth auscultation sound data, ninth enhanced auscultation sound data acquired by inputting the sixth auscultation sound data to the secondarily updated second artificial intelligence model, and score data corresponding to the ninth enhanced auscultation sound data.

The score data may include at least one of score data determined by a medical specialist in association with the ninth enhanced auscultation sound data, and score data acquired by inputting the ninth enhanced auscultation sound data to a perception-based loss function.

The tertiarily updating of the secondarily updated second artificial intelligence model may include updating the second artificial intelligence model so that a similarity between a first feature vector corresponding to the secondarily updated second artificial intelligence model and a second feature vector corresponding to the second artificial intelligence model to be tertiarily updated is present within a set range.

The artificial intelligence model training method may further include acquiring first sub-auscultation sound data corresponding to a bioacoustic sound and second sub-auscultation sound data corresponding to the noise by inputting seventh auscultation sound data to a third artificial intelligence model, and generating the second auscultation sound data by combining the first sub-auscultation sound data and the second sub-auscultation sound data, and the bioacoustic data may include the first sub-auscultation sound data.

A ratio between a first auscultation sound data set including the first auscultation sound data and a second auscultation sound data set including the second auscultation sound data may be determined to be a set value.

The artificial intelligence model training method may further include receiving eighth auscultation sound data from an external electronic apparatus, acquiring tenth enhanced auscultation sound data by inputting the eighth auscultation sound data to the second artificial intelligence model, and transmitting the tenth enhanced auscultation sound data to the external electronic apparatus.

The electronic apparatus may include a sound collection part, and a display, and the artificial intelligence model training method may further include acquiring eleventh enhanced auscultation sound data by inputting ninth auscultation sound data acquired through the sound collection part to the second artificial intelligence model, acquiring an abnormality analysis result corresponding to the eleventh enhanced auscultation sound data by inputting the eleventh enhanced auscultation sound data to a fourth artificial intelligence model, and providing the abnormality analysis result through the display.

According to another aspect, there is also provided an electronic apparatus including a memory, and a processor, and the processor is configured to primarily update a second artificial intelligence based on first auscultation sound data and output data of a first artificial intelligence model, update the first artificial intelligence model based on second auscultation sound data, bioacoustics data with noise removed from the second auscultation sound data, output data of the second artificial intelligence model, and output data of the primarily updated second artificial intelligence model, secondarily update the primarily updated second artificial intelligence model based on third auscultation sound data and output data of the updated first artificial intelligence model, and tertiarily update the secondarily updated second artificial intelligence model based on a reward corresponding to output data of the secondarily updated second artificial intelligence model for fourth auscultation sound data.

According to still another aspect, there is also provided a non-transitory computer-readable recording medium in which a program for executing the artificial intelligence model training method in a computer is recorded.

Additional aspects of example embodiments will be set forth in part in the description which follows and, in part, will be apparent from the description, or may be learned by practice of the disclosure.

According to example embodiments, an electronic apparatus may repeatedly perform a process of updating a second artificial intelligence model based on output data of a first artificial intelligence model and updating the first artificial intelligence model based on output data of the second artificial intelligence model. Through this, knowledge learned by the first artificial intelligence model may be further effectively transferred to the second artificial intelligence model.

According to example embodiments, in an environment in which securing correct answer data corresponding to auscultation sound data or bioacoustic data with noise removed from the auscultation sound data is difficult, the second artificial intelligence model may be trained by using unlabeled auscultation sound data of which an amount is larger than that of labeled auscultation sound data.

According to example embodiments, the electronic apparatus may train a reward model based on score data determined by a medical specialist or score data that is output by a perception-based loss function. Through this, the reward model may output a further accurate reward corresponding to enhanced auscultation sound data. Accordingly, the electronic apparatus may further effectively train an artificial intelligence model.

According to example embodiments, the electronic apparatus may limit a range of updating the artificial intelligence model to prevent convergence on a local minimum. Through this, the artificial intelligence model may be further effectively trained.

According to example embodiments, the electronic apparatus may generate training auscultation sound data by using the trained artificial intelligence model. Through this, a training auscultation sound data set in which corresponding correct answer data is present may be further conveniently and easily generated.

Effects of the present disclosure are not limited to those described above and other effects may be made apparent to those skilled in the art from the following description of the accompanying claims.

### BRIEF DESCRIPTION OF THE SEVERAL VIEWS OF THE DRAWINGS

These and/or other aspects, features, and advantages of the disclosure will become apparent and more readily appreciated from the following description of example embodiments, taken in conjunction with the accompanying drawings of which:

FIG. 1 illustrates a system according to an example embodiment;

FIG. 2 is a diagram for describing a method of an electronic apparatus training an artificial intelligence model according to an example embodiment;

FIG. 3 is a diagram for describing a method of an electronic apparatus training an artificial intelligence model according to an example embodiment;

FIG. 4 is a diagram for describing a method of an electronic apparatus training an artificial intelligence model according to an example embodiment;

FIG. 5 is a diagram for describing a method of an electronic apparatus training an artificial intelligence model according to an example embodiment;

FIGS. 6A through 6D illustrate an example of auscultation sound data, noise, and enhanced auscultation sound data according to an example embodiment;

FIGS. 7A through 7E are diagrams for describing a method of an electronic apparatus generating training auscultation sound data according to an example embodiment;

FIG. 8 is a flowchart illustrating an artificial intelligence model training method of an electronic apparatus according to an example embodiment; and

FIG. 9 is a block diagram illustrating an electronic apparatus according to an example embodiment.

### DETAILED DESCRIPTION

Terms used in the example embodiments are selected, as much as possible, from general terms that are widely used at present while taking into consideration the functions obtained in accordance with the present disclosure, but these terms may be replaced by other terms based on intentions of those skilled in the art, customs, emergence of new technologies, or the like. Also, in a particular case, terms that are arbitrarily selected by the applicant of the present disclosure may be used. In this case, the meanings of these terms may be described in corresponding description parts of the disclosure. Accordingly, it should be noted that the terms used herein should be construed based on practical meanings thereof and the whole content of this specification, rather than being simply construed based on names of the terms.

In the entire specification, when an element is referred to as “including” another element, the element should not be understood as excluding other elements so long as there is no special conflicting description, and the element may include at least one other element.

Throughout the specification, expression “at least one of a, b, and c” may include ‘a only’, ‘b only’, ‘c only’, ‘a and b’, ‘a and c’, ‘b and c’, or ‘all of a, b, and c’.

In the present disclosure, a “terminal” may be implemented as a computer or a portable terminal capable of accessing a server or another apparatus through a network. The computer may include, for example, a laptop computer, a desktop computer, and a notebook equipped with a web browser. The portable apparatus may be a wireless communication device ensuring a portability and a mobility, and include any type of handheld wireless communication device, for example, a tablet PC, a smartphone, a communication-based apparatus such as international mobile telecommunication (IMT), code division multiple access (CDMA), W-code division multiple access (W-CDMA), and long term evolution (LTE).

In the following description, example embodiments of the present disclosure will be described in detail with reference to the accompanying drawings so that those skilled in the art can easily carry out the present disclosure. However, the present disclosure may be embodied in many different forms and is not limited to the example embodiments described herein.

A function associated with an artificial intelligence according to the present disclosure is operated through a processor and a memory. The processor may be formed as one or a plurality of processors. At this point, the one or the plurality of processors may be a general-purpose processor such as a central processing unit (CPU), an application processor (AP), or a digital signal processor (DSP), a graphic-dedicated processor such as a graphics processing unit (GPU) or a vision processing unit (VPU), or an artificial intelligence-dedicated processor such as a neural processing unit (NPU). The one or the plurality of processors perform control so as to process input data according to a predefined operation rule stored in the memory or an artificial intelligence model. Alternatively, when the one or the plurality of processors are the artificial intelligence-dedicated processor, the artificial intelligence-dedicated processor may be designed in a hardware structure specializing in processing of a predetermined artificial intelligence model.

The predefined operation rule or the artificial intelligence model has a feature of being formed through learning. Here, being formed through the leaning may mean that a basic artificial intelligence model is trained with multiple pieces of learning data by a learning algorithm so that the predefined operation rule or the artificial intelligence model which is set to perform a desired characteristic (or purpose) is formed. Such training may be performed in an equipment itself in which the artificial intelligence according to the present disclosure is carried out or may be performed through an additional server and/or system. An example of the learning algorithm includes supervised learning, unsupervised learning, semi-supervised learning, or reinforcement learning, but it is merely an example.

The artificial intelligence (AI) model may be formed of a plurality of neural network layers. Each of the plurality of neural network layers has a plurality of weight values and performs neural network calculation through a calculation result of a previous layer and calculation between the plurality of weight values. The plurality of weight values of the plurality of neural network layers may be optimized by a result of training the artificial intelligence model. For example, the plurality of weight values may be renewed so that a loss value or a cost value acquired in the artificial intelligence model during the training is decreased or optimized. An artificial neural network may include a deep neural network (DNN), a convolutional neural network (CNN), a recurrent neural network (RNN), a restricted Boltzmann machine (RBM), a deep belief network (DBN), a bidirectional recurrent deep neural network (BRDNN), Deep Q-Networks, or the like, but it is merely an example.

Hereinafter, the example embodiments of the present disclosure will be described with reference to the accompanying drawings.

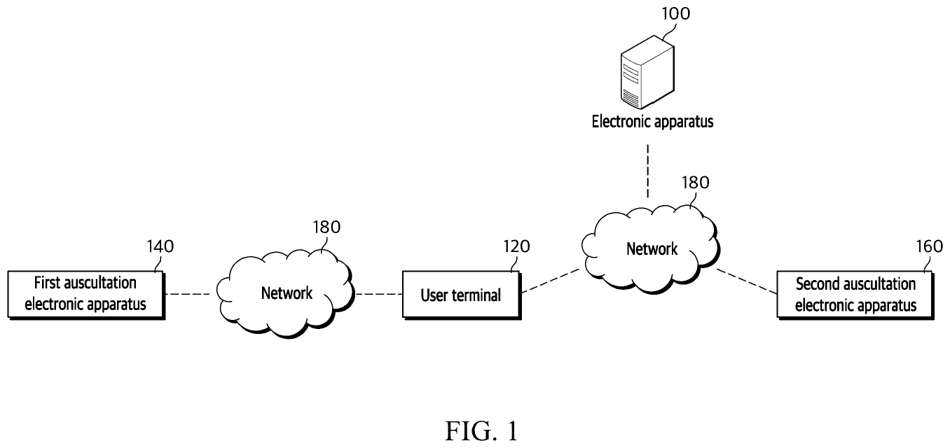

FIG. 1 illustrates a system according to an example embodiment.

Referring to FIG. 1, the system may include at least one of an electronic apparatus 100, a user terminal 120, one or more auscultation electronic apparatuses 140 and 160, and a network 180. Meanwhile, FIG. 1 illustrates only elements associated with the present example embodiment among elements included in the system. Thus, those skilled in the art associated with the present example embodiment may understand that other elements in general use in addition to the elements illustrated in FIG. 1 may be further included.

The electronic apparatus 100 is an apparatus performing various functions associated with an artificial intelligence model outputting enhanced auscultation sound data based on auscultation sound data. For example, the electronic apparatus 100 may train the artificial intelligence model based on the auscultation sound data and bioacoustic data with noise removed from the auscultation sound data. Alternatively, the electronic apparatus 100 may receive the auscultation sound data from the user terminal 120 or a second auscultation electronic apparatus 160, acquire the enhanced auscultation sound data by inputting the received auscultation sound data to the artificial intelligence model, and transmit the acquired enhanced auscultation sound data to the user terminal 120 or the second auscultation electronic apparatus 160.

The user terminal 120 may be a terminal used by each user, and users may use respective user terminals 120 to access a service provided by the network 180. More specifically, the electronic apparatus 100 may provide an application for diagnosing a disease of a user to the user terminal 120. The users may use the application, which is installed in the respective user terminals 120, to measure an auscultation sound and receive an enhanced auscultation sound or a disease diagnosis result according thereto.

As an example, the user terminal 120 may receive auscultation sound data received by a first auscultation electronic apparatus 140 and transmit the received auscultation sound data to the electronic apparatus 100. The electronic apparatus 100 may acquire the enhanced auscultation sound data based on the auscultation sound data and transmit the acquired enhanced auscultation sound data to the user terminal 120. Afterward, the user terminal 120 may provide the enhanced auscultation sound data through a display or a sound output part.

As another example, the user terminal 120 may receive the auscultation sound data received by the first auscultation electronic apparatus 140 and transmit the received auscultation sound data to the electronic apparatus 100. The electronic apparatus 100 may acquire the enhanced auscultation sound data based on the auscultation sound data and transmit the acquired improved auscultation sound data to the user terminal 120. Afterward, the user terminal 120 may transmit the enhanced auscultation sound data to the first auscultation electronic apparatus 140, and the first auscultation electronic apparatus 140 may provide the enhanced auscultation sound data through a display or a sound output part.

As another example, the user terminal 120 may receive the auscultation sound data received by the first auscultation electronic apparatus 140 and transmit the received auscultation sound data to the electronic apparatus 100. The electronic apparatus 100 may acquire the enhanced auscultation sound data based on the auscultation sound data and transmit the acquired enhanced auscultation sound data to the user terminal 120. Afterward, the user terminal 120 may transmit the enhanced auscultation sound data to the first auscultation electronic apparatus 140, and the first auscultation electronic apparatus 140 may provide the disease diagnosis result, which is acquired based on the enhanced auscultation sound data, through the display or the sound output part.

As another example, the user terminal 120 may receive auscultation sound data acquired by the first auscultation electronic apparatus 140 and transmit the received auscultation sound data to the electronic apparatus 100. The electronic apparatus 100 may acquire the enhanced auscultation sound data based on the auscultation sound data and acquire the disease diagnosis result based on the acquired enhanced auscultation sound data. Afterward, the electronic apparatus 100 may provide the disease diagnosis result through the application installed in the user terminal 120.

The one or more auscultation electronic apparatuses 140 and 160 is an apparatus performing a function of acquiring the auscultation sound data from a subject to be examined. For example, a user may allow a sound collection part of the one or more auscultation electronic apparatuses 140 and 160 to be in contact with skin or clothes on a body region of the subject to be examined. The one or more auscultation electronic apparatuses 140 and 160 may acquire vibration and a sound transferred through the skin or the clothes and acquire the auscultation sound data based thereon. In addition, the one or more auscultation electronic apparatuses 140 and 160 may acquire the enhanced auscultation sound data based on the acquired auscultation sound data and provide the enhanced auscultation sound data through a display or a sound output part or acquire an abnormality analysis result based on the auscultation sound data and provide the abnormality analysis result through the display.

Meanwhile, although FIG. 1 illustrates the electronic apparatus 100 as an apparatus different from the one or more auscultation electronic apparatuses 140 and 160, the electronic apparatus 100 may represent an apparatus performing a function identical to that of the one or more auscultation electronic apparatuses 140 and 160. For example, the electronic apparatus 100 may include a sound collection part and a display. Accordingly, the electronic apparatus 100 may acquire the auscultation sound data through the sound collection part, acquire the enhanced auscultation sound data based on the auscultation sound data, and provide the abnormality analysis data which corresponds to the enhanced auscultation sound data through the display.

The user terminal 120, the second auscultation electronic apparatus 160, and the electronic apparatus 100 may, or the user terminal 120 and the first auscultation electronic apparatus 140 may communicate with each other in the network 180. The network 180 may include a local area network (LAN), a wide area network (WAN), a value added network (VAN), a mobile radio communication network, a satellite communication network, and a combination thereof, may be a comprehensive data network for allowing each network entity illustrated in FIG. 1 to smoothly communicate with another, and may include the wired Internet, the wireless Internet, and a mobile wireless communication network. Wireless communication may include, for example, a wireless LAN (e.g., Wireless Fidelity (Wi-Fi)), Bluetooth, Bluetooth low energy, ZigBee, Wi-Fi Direct (WFD), ultra wideband (UWB), Infrared Data Association (IrDA), near field communication (NFC), or the like. However, it is merely an example.

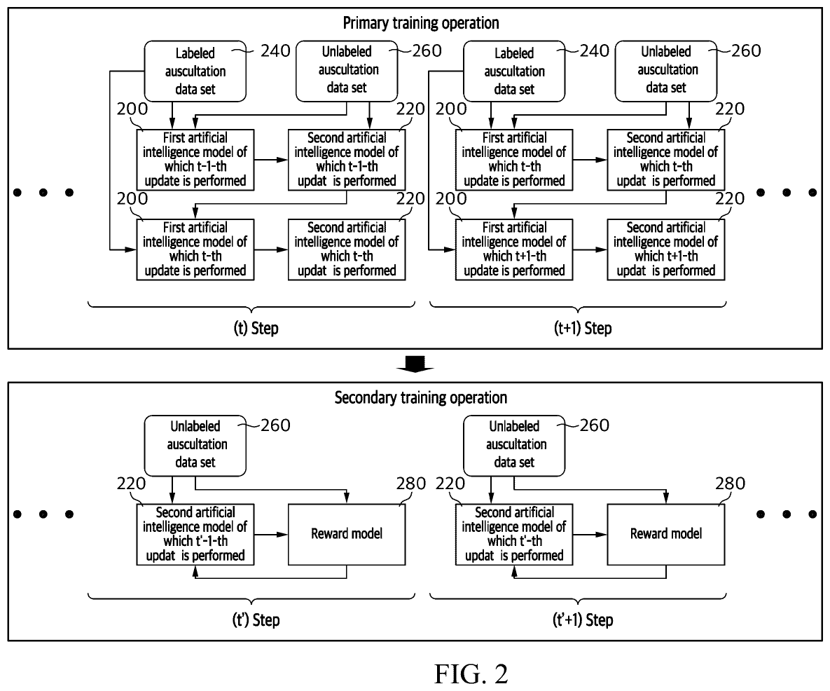

FIG. 2 is a diagram for describing a method of the electronic apparatus 100 training an artificial intelligence model according to an example embodiment. At this point, a rounded quadrilateral represents data or information.

According to an example embodiment, the electronic apparatus 100 may train, based on a labeled auscultation sound data set 240 or an unlabeled auscultation sound data set 260, the artificial intelligence model which outputs enhanced auscultation sound data. More specifically, the electronic apparatus 100 may perform a primary training operation for a second artificial intelligence model 220 and then perform a secondary training operation for the second artificial intelligence model 220 for which primary training is completed.

For example, the electronic apparatus 100 may simultaneously train a first artificial intelligence model 200 and the second artificial intelligence model 220 based on the labeled auscultation sound data set 240 and the unlabeled auscultation sound data set 260. Afterward, the electronic apparatus 100 may train the second artificial intelligence model 220 based on the unlabeled auscultation sound data set 260 and a reward model 280.

At this point, the labeled auscultation sound data set 240 may be an auscultation sound data set in which corresponding correct answer data is present and that includes noise, and the unlabeled auscultation sound data set 260 may be an auscultation sound data set in which the corresponding correct answer data is absent and that includes the noise. Also, the correct answer data may be target data that the first artificial intelligence model 200 or the second artificial intelligence model 220 may finally output based on auscultation sound data and may be data in which only a bioacoustics sound (e.g., a heart sound or a lung sound) is present as noise is removed from the auscultation sound data. However, a term referring to labeled auscultation sound data, unlabeled auscultation sound data, or the correct answer data is not limited the above description.

According to an example embodiment, the electronic apparatus 100 may perform the primary training operation for the second artificial intelligence model 220 based on the labeled auscultation sound data set 240 and the unlabeled auscultation sound data set 260. More specifically, the electronic apparatus 100 may repeatedly perform a process of simultaneously training the first artificial intelligence model 200 and the second artificial intelligence model 220 based on the labeled auscultation sound data set 240 and the unlabeled auscultation sound data set 260.

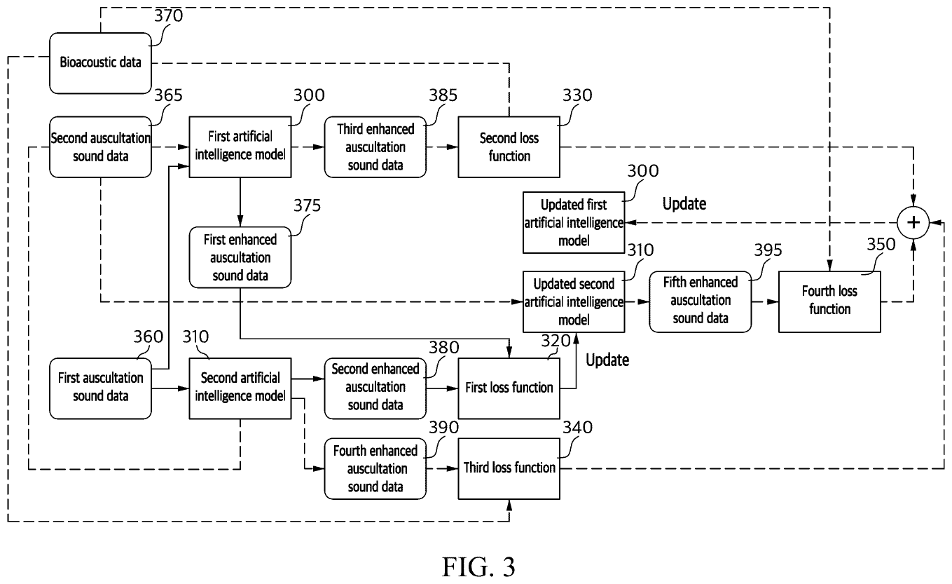

For example, in a t-th step, the electronic apparatus 100 may perform, based on the unlabeled auscultation sound data set 260 and output data of the first artificial intelligence model 200 of which a t−1-th update is performed, a t-th update of the second artificial intelligence model 220 of which a t−1-th update is performed. Afterward, the electronic apparatus 100 may perform, based on the labeled auscultation sound data set 240, output data of the second artificial intelligence model 220 of which the t−1-th update is performed, and output data of the second artificial intelligence model 220 of which the t-th update is performed, a t-th update of the first artificial intelligence model 200 of which the t−1-th update is performed. The electronic apparatus 110 may repeatedly perform such a process. A detailed process in which the electronic apparatus 100 performs the primary training operation for the second artificial intelligence model 220 will be described in detail with reference to FIG. 3.

At this point, the first artificial intelligence model 200 may be an artificial intelligence model trained in advance based on the labeled auscultation sound data set 240 and referred to as a teacher model, a t-model, or the like. The second artificial intelligence model 220 may be a model that receives knowledge learned by the first artificial intelligence model 200 and referred to as a student model, an s-model, a base model, or the like. Generally, the second artificial intelligence model 220 may represent a model smaller than the first artificial intelligence model 200, but it is merely an example.

According to an example embodiment, the electronic apparatus 100 may perform, based on the unlabeled auscultation sound data set 260 and the reward model 280, the secondary training operation for the second artificial intelligence model 220 for which the primary training is completed. More specifically, the electronic apparatus 100 may repeatedly perform a process of training the second artificial intelligence model 220 based on the unlabeled auscultation sound data set 260 and the reward model 280.

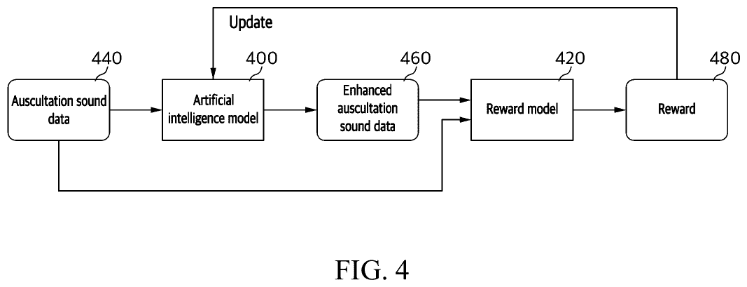

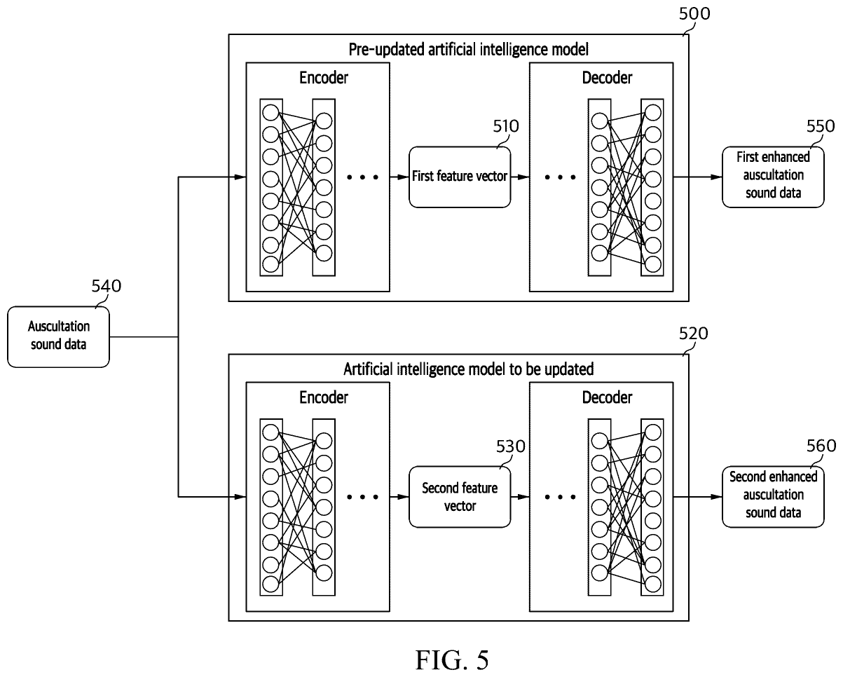

For example, in the t′-th step, the electronic apparatus 100 may acquire, based on the unlabeled auscultation sound data set 260 and output data, for the unlabeled auscultation sound data set 260, of the second artificial intelligence model 220 of which a t′−1-th update is performed, a reward that is output by the reward model 280. Afterward, the electronic apparatus 100 may perform, based on the reward, the t′-th update of the second artificial intelligence model 220 of which the t′−1-th update is performed. The electronic apparatus 110 may repeatedly perform such a process. A detailed process in which the electronic apparatus 100 performs the secondary training operation for the second artificial intelligence model 220 will be described in detail with reference to FIGS. 4 and 5.

FIG. 3 is a diagram for describing a method of the electronic apparatus 100 training an artificial intelligence model according to an example embodiment. Redundant descriptions similar to those of FIG. 2 will be simplified or omitted.

According to an example embodiment, the electronic apparatus 100 may train a first artificial intelligence model 300 and a second artificial intelligence model 310 based on first auscultation sound data 360, second auscultation sound data 365, and bioacoustics data 370. For example, the electronic apparatus 100 may update the second artificial intelligence model 310 based on the first auscultation sound data 360 and output data of the first artificial intelligence model 300. Afterward, the electronic apparatus 100 may update the first artificial intelligence model 300 based on the second auscultation sound data 365, the bioacoustics data 370 with noise removed from the second auscultation sound data 365, output data of the second artificial intelligence model 310, and output data of the updated second artificial intelligence model 310.

At this point, the first artificial intelligence model 300 of FIG. 3 may correspond to the first artificial intelligence model 200 of FIG. 2, and the second artificial intelligence model 310 of FIG. 3 may correspond to the second artificial intelligence model 220 of FIG. 2. In addition, the first auscultation sound data 360 of FIG. 3 may be included in the unlabeled auscultation sound data set 260 of FIG. 2. The second auscultation sound data 365 of FIG. 3 and the bioacoustics data 370 may be included in the labeled auscultation sound data set 240 of FIG. 2. However, it is merely an example.

According to an example embodiment, the electronic apparatus 100 may update the second artificial intelligence model 310 based on the first auscultation sound data 360 and the output data of the first artificial intelligence model 300.

More specifically, referring to FIG. 3, the electronic apparatus 100 may acquire first enhanced auscultation sound data 375 by inputting the first auscultation sound data 360 to the first artificial intelligence model 300 and acquire second enhanced auscultation sound data 380 by inputting the first auscultation sound data 360 to the second artificial intelligence model 310. Afterward, the electronic apparatus 100 may acquire a first loss by inputting the first enhanced auscultation sound data 375 and the second enhanced auscultation sound data 380 to a first loss function 320 and update the second artificial intelligence model 310 so that the first loss is minimized. As such, a line associated with a process of updating the second artificial intelligence model 310 may be shown as a solid line in FIG. 3.

For example, in a t-th step, the electronic apparatus 100 may acquire the first enhanced auscultation sound data 375 which is denoted by Et, u(t−1) by inputting the first auscultation sound data 360 which is denoted by NU to the first artificial intelligence model 300 of which a t−1-th update is performed and which is denoted by Mt(t−1) and acquire the second enhanced auscultation sound data 380 which is denoted by Es, u(t−1) by inputting the first auscultation sound data 360 to the second artificial intelligence model 310 of which a t−1-th update is performed and which is denoted by Ms(t−1). Afterward, the electronic apparatus 100 may acquire the first loss which is denoted by Ls, u(t−1) by inputting the first enhanced auscultation sound data 375 and the second enhanced auscultation sound data 380 to the first loss function 320 which is denoted by L1 and update the second artificial intelligence model 310, of which the t−1-th update is performed, to be the second artificial intelligence model 310 of which a t-th update is perform and which is denoted by Mst so that the first loss is minimized. Such an operation may be represented by the following equation.
Acquire the first enhanced auscultation sound data 375: Mₜ^{(t-1)}(N_{U})→E_{t, u}^{(t-1)}
Acquire the second enhanced auscultation sound data 380: Mₛ^{(t-1)}(N_{U})→E_{s, u}^{(t-1)}
Acquire the first loss: L₁(E_{t, u}^{(t-1)}, E_{s, u}^{(t-1)})→L_{s, u}^{(t-1)}
Update the second artificial intelligence model 310: update (Mₛ^{(t-1)}, L_{s, u}^{(t-1)})→Mst

According to an example embodiment, the electronic apparatus 100 may update the first artificial intelligence model 300 based on the second auscultation sound data 365, the bioacoustics data 370 with the noise removed from the second auscultation sound data 365, the output data of the second artificial intelligence model 310, and the output data of the updated second artificial intelligence model 310.

More specifically, referring to FIG. 3, the electronic apparatus 100 may acquire third enhanced auscultation sound data 385 by inputting the second auscultation sound data 365 to the first artificial intelligence model 300, acquire fourth enhanced auscultation sound data 390 by inputting the second auscultation sound data 365 to the second artificial intelligence model 310, and acquire fifth enhanced auscultation sound data 395 by inputting the second auscultation sound data 365 to the updated second artificial intelligence model 310. Afterward, the electronic apparatus 100 may acquire a second loss by inputting the third enhanced auscultation sound data 385 and the bioacoustics data 370 to a second loss function 330, acquire a third loss by inputting the fourth enhanced auscultation sound data 390 and the bioacoustic data 370 to a third loss function 340, and acquire a fourth loss by inputting the fifth enhanced auscultation sound data 395 and the bioacoustics data 370 to a fourth loss function 350. The electronic apparatus 100 may update the first artificial intelligence model 300 so that a sum of the second loss, the third loss, and the fourth loss is minimized. As such, a line associated with a process of updating the first artificial intelligence model 300 may be shown as a dotted line in FIG. 3.

For example, in the t-th step, the electronic apparatus 100 may acquire the third enhanced auscultation sound data 385 which denoted by Et, l(t−1) by inputting the second auscultation sound data 365 which is denoted by Nl to the first artificial intelligence model 300 of which the t−1-th update is performed and which is denoted denoted by Mt(t−1), acquire the fourth enhanced auscultation sound data 390 which is denoted by Es, l(t−1) by inputting the second auscultation sound data 365 to the second artificial intelligence model 310 of which the t−1-th update is performed and which is denoted by Ms(t−1), and acquire the fifth auscultation sound data 395 which is denoted by Es, lt by inputting the second auscultation sound data 365 to the updated second artificial intelligence model 310 of which the t-th update is performed and which is denoted by Mst. Afterward, the electronic apparatus 100 may acquire the second loss which is denoted by Lt, l(t−1) by inputting the third enhanced auscultation sound data 385 and the bioacoustics data 370 which is denoted by Cl to the second loss function 330 which is denoted by L2, acquire the third loss which is denoted by Ls, l(t−1) by inputting the fourth enhanced auscultation sound data 390 and the bioacoustic data 370 to the third loss function 340 which is denoted by L3, and acquire the fourth loss which is denoted by Ls, lt by inputting the fifth auscultation sound data 395 and the bioacoustics data 370 to the fourth loss function 350 which is denoted by L4. The electronic apparatus 100 may update the first artificial intelligence model 300, of which the t−1-th update is performed, to be the first artificial intelligence model 300 of which a t-th update is performed and which is denoted by Mtt so that the sum of the second loss, the third loss, and the fourth loss is minimized. Such an operation may be represented by the following equation.
Acquire the third enhanced auscultation sound data 385: Mₜ^{(t-1)}(Nₗ)→E_{t, l}^{(t-1)}
Acquire the fourth enhanced auscultation sound data 390: Mₛ^{(t-1)}(Nₗ)→E_{s, l}^{(t-1)}
Acquire the fifth enhanced auscultation sound data 395: Mₛ^{t}(Nₗ)→E_{s, l}^{t}
Acquire the second loss: L₂(E_{t, l}^{(t-1)}, Cₗ)→L_{t, l}^{(t-1)}
Acquire the third loss: L₃(E_{s, l}^{(t-1)}, Cₗ)→L_{s, l}^{(t-1)}
Acquire the fourth loss: L₄(E_{s, l}^{t}, Cₗ)→L_{s, l}^{t}
Update the first artificial intelligence model 300: update (Mₜ^{(t-1)}, L_{t, l}^{(t-1)}+L_{s, l}^{(t-1)}+L_{s, l}^{t})→>Mₜ^{t}

As such, the electronic apparatus 100 may repeatedly perform a process of updating the second artificial intelligence model 310 based on the output data of the first artificial intelligence model 300 and updating the first artificial intelligence model 300 based on the output data of the second artificial intelligence model 310. Through this, knowledge learned by the first artificial intelligence model 300 may be further effectively transferred to the second artificial intelligence model 310. Also, in an environment in which securing correct answer data corresponding to auscultation sound data or bioacoustic data with noise removed from the auscultation sound data is difficult, the second artificial intelligence model 310 may be trained by using unlabeled auscultation sound data of which an amount is larger than that of labeled auscultation sound data.

According to an example embodiment, the electronic apparatus 100 may determine a ratio between a first auscultation sound data set including the first auscultation sound data 360 and a second auscultation sound data set including the second auscultation sound data 365 to be a set value. For example, the electronic apparatus 100 may draw an optimal training result by determining the ratio between the first auscultation sound data set and the second auscultation sound data set to be 1:4.

FIG. 4 is a diagram for describing a method of the electronic apparatus 100 training an artificial intelligence model according to an example embodiment. Redundant descriptions similar to those of FIG. 2 will be simplified or omitted.

According to an example embodiment, the electronic apparatus 100 may train an artificial intelligence model 400 based on auscultation sound data 440 and a reward model 420. For example, the electronic apparatus 100 may acquire enhanced auscultation sound data 460 by inputting the auscultation sound data 440 to the artificial intelligence model 400. Afterward, the electronic apparatus 100 may acquire a reward 480 by inputting the auscultation sound data 440 and the enhanced auscultation sound data 460 and update the artificial intelligence model 400 so that the reward 480 is maximized.

At this point, the first artificial intelligence model 400 of FIG. 4 may correspond to the second artificial intelligence model 220 of FIG. 2, and the reward model 420 of FIG. 4 may correspond to the reward model 280 of FIG. 2. In addition, the auscultation sound data 440 of FIG. 4 may be included in the unlabeled auscultation sound data set 260 of FIG. 2, but it is merely an example.

According to an example embodiment, the electronic apparatus 100 may train the artificial intelligence model 400 based on reinforcement learning. For example, the electronic apparatus 100 may set the artificial intelligence model 400 to be an agent for performing the reinforcement learning, set the artificial intelligence 400 outputting the enhanced auscultation sound data 460 based on the auscultation sound data 440 to be an action of the agent, and set a reward according to the action of the agent based on a score for the enhanced auscultation sound data 460 which is output by the reward model 420. Afterward, the electronic apparatus 100 may update the artificial intelligence model 400 so that the reward according to the action of the agent is maximized.

However, it is merely an example, and the electronic apparatus 100 may set a probability that the artificial intelligence model 400 takes a predetermined action to be a policy. A scheme of the electronic apparatus 100 setting a parameter in order to perform the reinforcement learning is not limited to the above description.

According to an example embodiment, when training the artificial intelligence model 400, the electronic apparatus 100 may limit a range of updating the artificial intelligence model 400. For example, the electronic apparatus 100 may update the artificial intelligence model 400 so that a similarity between a first feature vector corresponding to the artificial intelligence model 400 and a second feature vector corresponding to the artificial intelligence model 400 to be updated is present within a set range. A detailed process in which the artificial intelligence 100 limits the range of updating the artificial intelligence model 400 will be described in detail with reference to FIG. 5.

According to an example embodiment, the electronic apparatus 100 may train the reward model 420 based on the auscultation sound data 440, the enhanced auscultation sound data 460, and score data corresponding to the enhanced auscultation sound data 460. For example, the electronic apparatus 100 may supervised-learn the reward model 420 with, as target data, at least one of score data determined by a medical specialist in association with the enhanced auscultation sound data 460 and score data acquired by inputting the enhanced auscultation sound data 460 to a perception-based loss function. At this point, the perception-based loss function may include a perceptual metric for speech quality evaluation (PMSQE) loss function or a log mel spectral (LMS) loss function, but it is merely an example.

Meanwhile, the reward model 420 may represent the perception-based loss function itself in addition to an artificial intelligence model trained based the auscultation sound data 440, the enhanced auscultation sound data 460, and the score data corresponding to the enhanced auscultation sound data 460.

As such, the electronic apparatus 100 may train the reward model 420 based on the score data determined by the medical specialist or the score data which is output by the perception-based loss function, and through this, the reward model 420 may output the reward 480 which corresponds to the enhanced auscultation sound data 460 and is further accurate. Accordingly, the electronic apparatus 100 may further effectively train the artificial intelligence model 400.

FIG. 5 is a diagram for describing a method of the electronic apparatus 100 training an artificial intelligence model according to an example embodiment. Redundant descriptions similar to those of FIGS. 2 and 4 will be simplified or omitted.

According to an example embodiment, the electronic apparatus 100 may limit a range of updating the artificial intelligence model to train the artificial intelligence model. For example, the electronic apparatus 100 may update the artificial intelligence model based on a trust region policy optimization (TRPO) algorithm so that a difference between a pre-update artificial intelligence model 500 and an artificial intelligence model 520 to be updated is within a set range.

At this point, the pre-update artificial intelligence model 500 of FIG. 5 may correspond to the second artificial intelligence model 220 of FIG. 2 of which a t′−1-th update is performed, and the artificial intelligence model 520 to be updated of FIG. 5 may correspond to the second artificial intelligence model 220 of FIG. 2 of which a t′-th update is performed. In addition, auscultation sound data 540 of FIG. 5 may be included in the unlabeled auscultation sound data set 260 of FIG. 2 or correspond to the auscultation sound data 440 of FIG. 4. First enhanced auscultation sound data 550 of FIG. 5 or second enhanced auscultation sound data 560 may correspond to the enhanced auscultation sound data 460 of FIG. 4. However, it is merely an example.

According to an example embodiment, the electronic apparatus 100 may update the artificial intelligence model so that a similarity between a first feature vector 510 corresponding to the pre-update artificial intelligence model 500 and a second feature vector 530 corresponding to the artificial intelligence model 520 to be updated is present within a set range.

For example, the electronic apparatus 100 may identify a cosine similarity between the first feature vector 510 and the second feature vector 530 based on the following Equation 1. Afterward, the electronic apparatus 100 may identify, based on the following Equations 2 and 3, the second feature vector 530 which may maximize a reward as far as the cosine similarity is present within a set threshold value.

$${sine}_{similarity ( A , B )} = \frac{A \cdot B}{{‖ A ‖}_{2} \times {‖ B ‖}_{2}} \qquad [ Equation 1 ]$$

$$clip ( cosine\_similarity ( A , B ) , {th}_{1} , {th}_{2} ) = \{ \begin{matrix}{th}_{2} , & cosine\_similarity ( A , B ) \geq {th}_{2} \\ {th}_{1} , & cosine\_similarity ( A , B ) \leq {th}_{1} \\ cosine\_similarity ( A , B ) , & otherwise\end{matrix} \qquad [ Equation 2 ]$$

$$B ← arg {max}_{B} E [ min ( cosine\_similarity ( A , B ) R , clip ( cosine\_similarity ( A , B ) , {th}_{1} , {th}_{2} ) R ) ] \qquad [ Equation 3 ]$$

At this point, A may denote the first feature vector 510, B may denote the second feature vector 530, and R may denote the reward. Also, th1 and th2 may denote threshold values which is set for the cosine similarity.

Meanwhile, in a process in which the auscultation sound data 540 is input to the artificial intelligence model and the enhanced auscultation sound data 550 is output, a feature vector may be a vector that is output from an encoder and input to a decoder and show a characteristic of the artificial intelligence model irrespective of the input auscultation sound data 540. For example, referring to FIG. 5, each of the pre-update artificial intelligence model 500 and the artificial intelligence model 520 to be updated may include an encoder formed of one or more layers and a decoder formed of one or more layers. The first feature vector 510 and the second feature vector 530 may represent vectors that are output from the encoder and input to the decoder. However, this is merely an example embodiment. The artificial intelligence model may have a structure other than an encoder-decoder structure illustrated in FIG. 5. Even in this case, the electronic apparatus 100 may identify a feature vector corresponding to the artificial intelligence model.

As such, the electronic apparatus 100 may limit the range of updating the artificial intelligence model to prevent convergence on a local minimum. Through this, the artificial intelligence model may be further effectively trained.

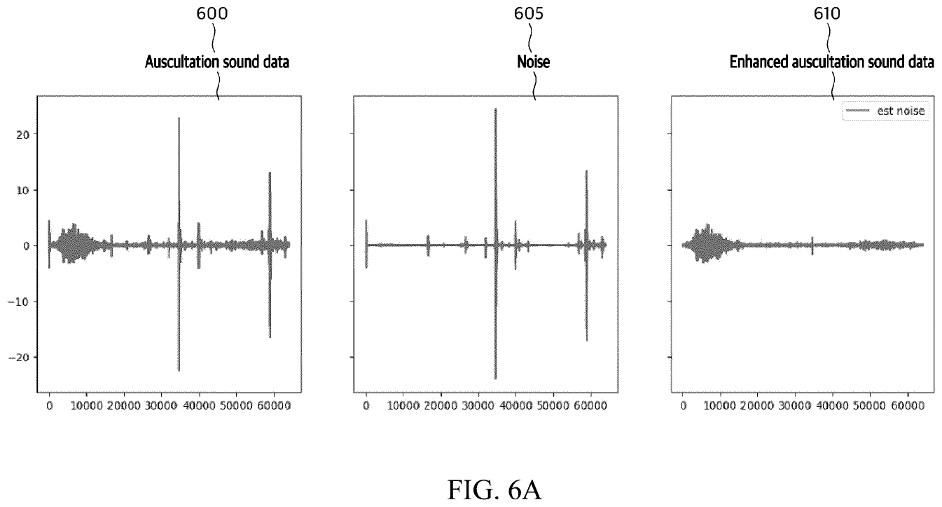

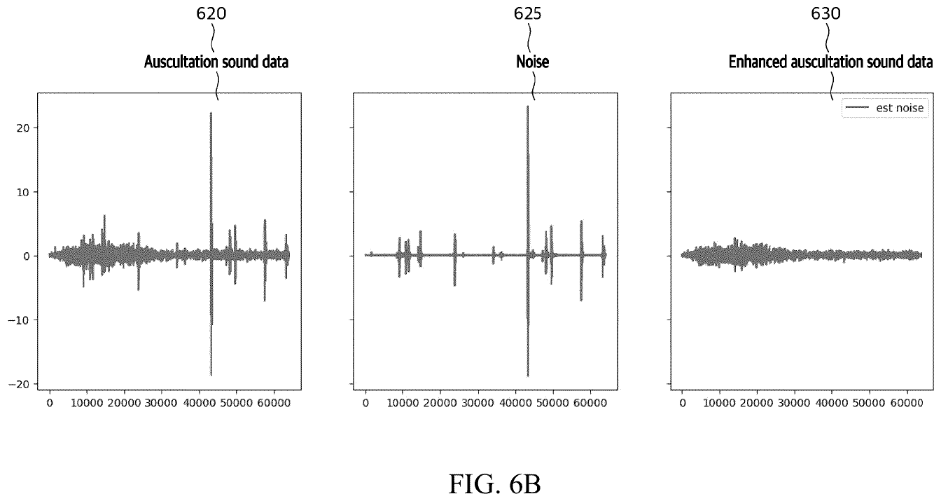

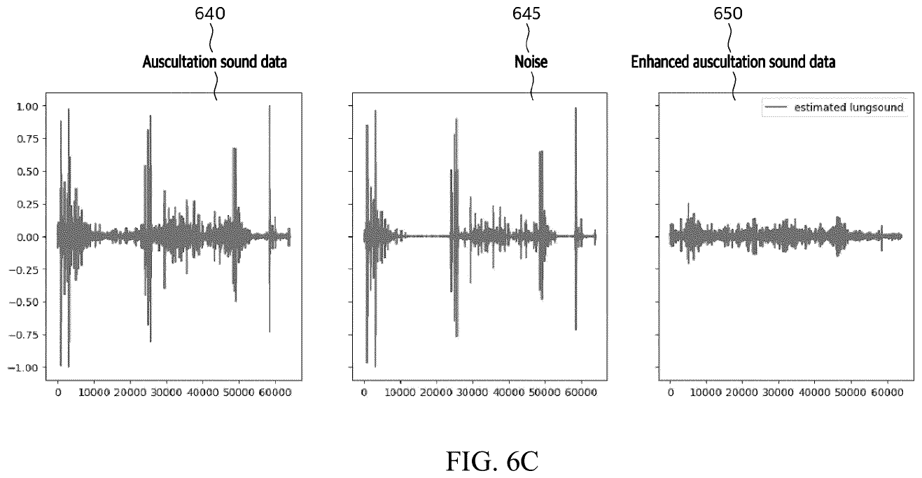

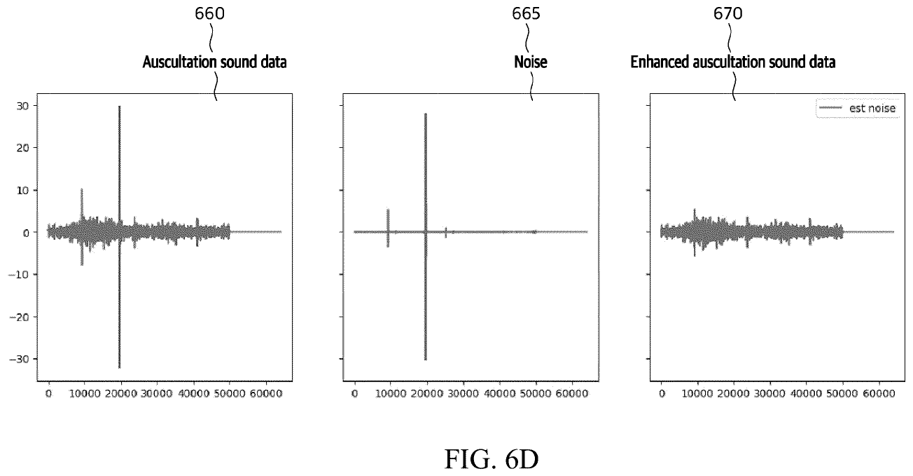

FIGS. 6A through 6D illustrate an example of auscultation sound data, noise, and enhanced auscultation sound data according to an example embodiment.

According to an example embodiment, the electronic apparatus 100 may acquire the enhanced auscultation sound data from the auscultation sound data by using a trained artificial intelligence model. More specifically, the electronic apparatus 100 may acquire the enhanced auscultation sound data with the noise removed by inputting the auscultation sound data to the artificial intelligence model.

As an example, referring to FIG. 6A, the electronic apparatus 100 may acquire enhanced auscultation sound data 610 with noise 605 removed by inputting auscultation sound data 600 to the trained artificial intelligence model.

As another example, referring to FIG. 6B, the electronic apparatus 100 may acquire enhanced auscultation sound data 630 with noise 625 removed by inputting auscultation sound data 620 to the trained artificial intelligence model.

As still another example, referring to FIG. 6C, the electronic apparatus 100 may acquire enhanced auscultation sound data 650 with noise 645 removed by inputting auscultation sound data 640 to the trained artificial intelligence model.

As still another example, referring to FIG. 6D, the electronic apparatus 100 may acquire enhanced auscultation sound data 670 with noise 665 removed by inputting auscultation sound data 660 to the trained artificial intelligence model.

According to an example embodiment, the electronic apparatus 100 may provide an abnormality analysis result corresponding to the enhanced auscultation sound data. For example, the electronic apparatus 100 may acquire the abnormality analysis result by inputting the enhanced auscultation sound data to the artificial intelligence model which is trained based on a learning data set including one or more pieces of auscultation sound data and one or more pieces of disease data. Afterward, the electronic apparatus 100 may provide the abnormality analysis result through a display.

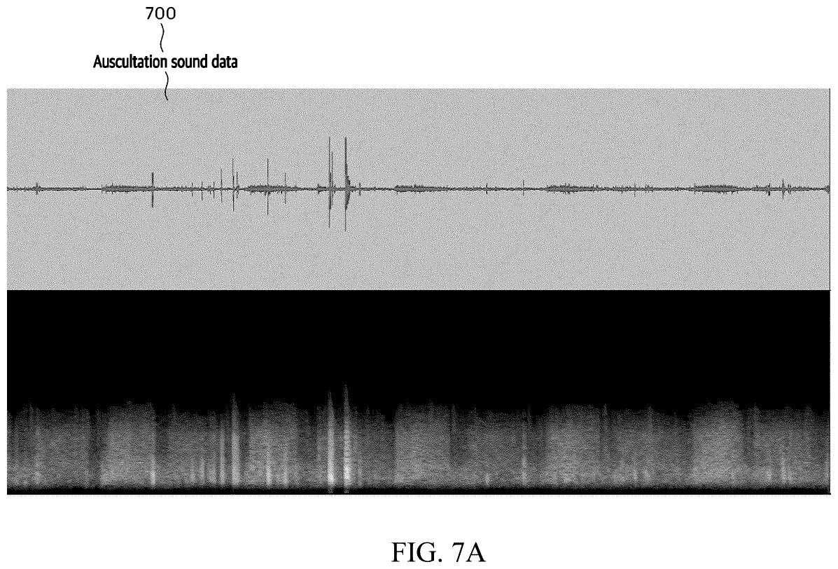

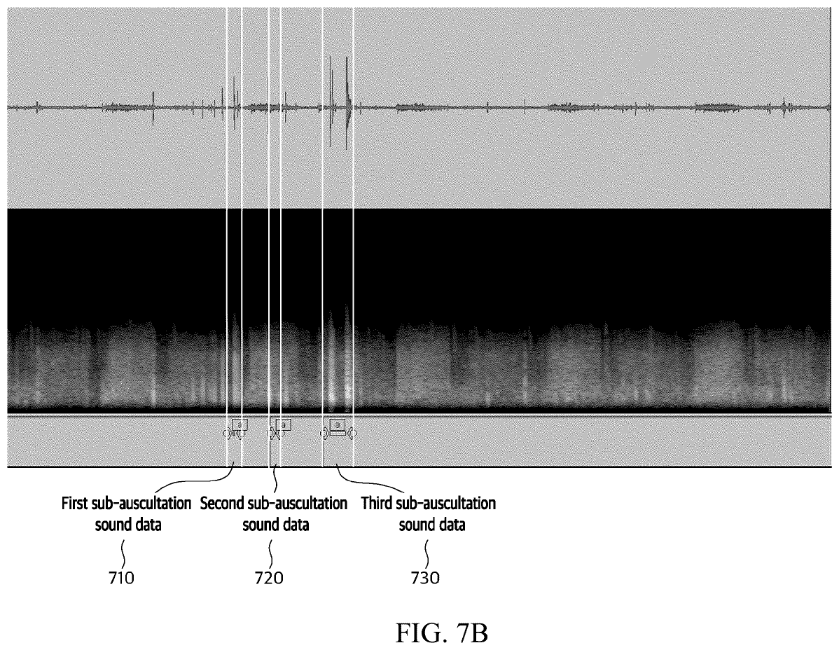

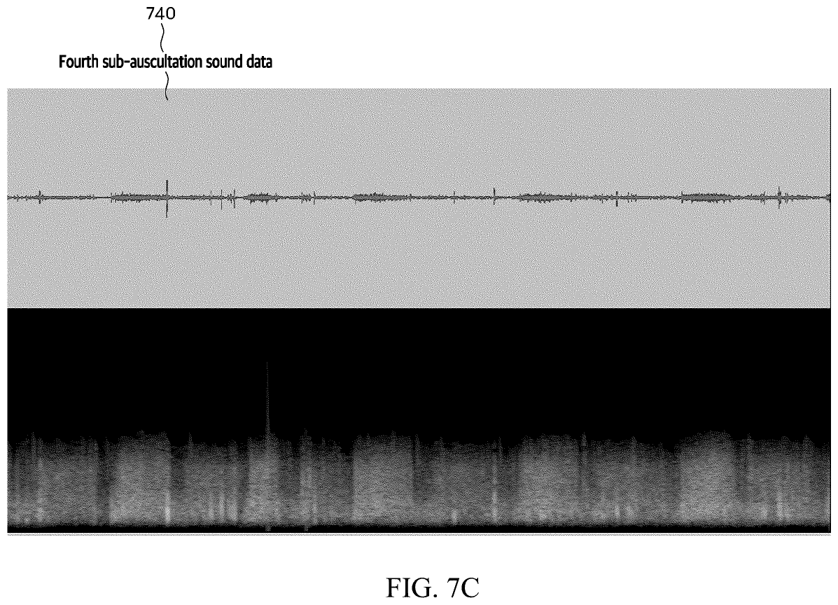

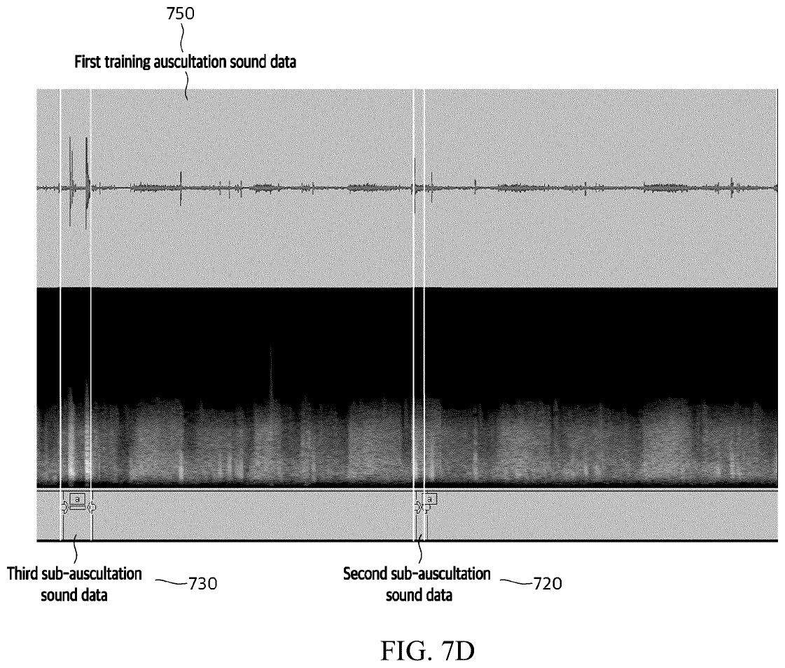

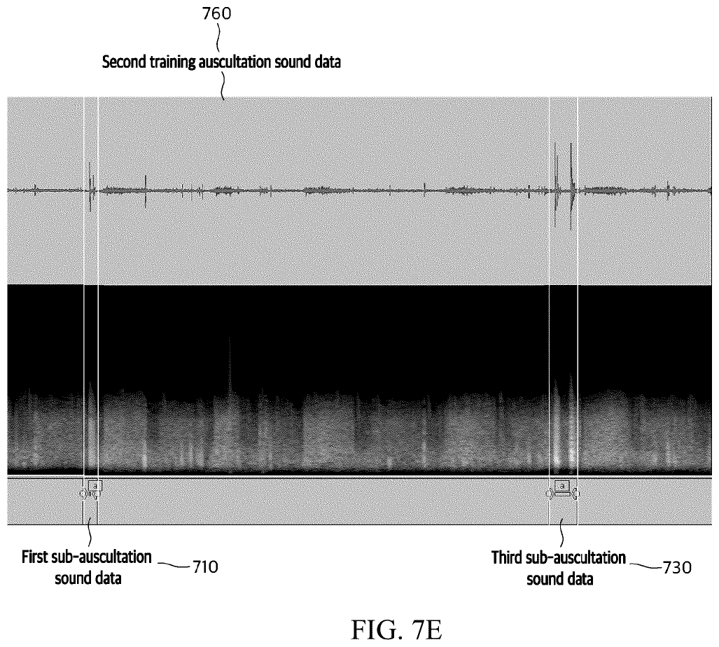

FIGS. 7A through 7E are diagrams for describing a method of the electronic apparatus 100 generating auscultation sound data for training according to an example embodiment.

According to an example embodiment, the electronic apparatus 100 may train an artificial intelligence model for distinguishing bioacoustics data or noise in auscultation sound data. For example, the electronic apparatus 100 may supervised-learn the artificial intelligence model with, as target data, the auscultation sound data in which an annotation is shown in each of the bioacoustics data and the noise which are included in the auscultation sound data. The electronic apparatus 100 may repeatedly perform a process of supervising and training the artificial intelligence model until an output value of the trained artificial intelligence model reaches a set reference.

According to an example embodiment, the electronic apparatus 100 may acquire, from the auscultation sound data, sub-auscultation sound data corresponding to a bioacoustic sound and sub-auscultation sound data corresponding to the noise by using the trained artificial intelligence model. More specifically, the electronic apparatus 100 may acquire the sub-auscultation sound data corresponding to the bioacoustic sound and the sub-auscultation sound data corresponding to the noise by inputting the auscultation sound data to the trained artificial intelligence model.

For example, referring to FIGS. 7A and 7B, the electronic apparatus 100 may acquire first sub-auscultation sound data 710, second sub-auscultation sound data 720, and third sub-auscultation sound data 730 that correspond to noise by inputting auscultation sound data 700 to the artificial intelligence model. In addition, referring to FIGS. 7A through 7C, the electronic apparatus 100 may acquire fourth sub-auscultation sound data 740 corresponding to a bioacoustic sound by inputting the auscultation sound data 700 to the artificial intelligence model.

At this point, the artificial intelligence model may distinguish the bioacoustic sound or the noise included in the auscultation sound data over time. Accordingly, the fourth sub-auscultation sound data 740 may represent temporally reduced auscultation sound data by excluding the first sub-auscultation sound data 710, the second sub-auscultation sound data 720, and the third sub-auscultation sound data 730 from the auscultation sound data 700.

According to an example embodiment, the electronic apparatus 100 may generate the auscultation sound data for the training by combining pieces of sub-auscultation sound data. More specifically, the electronic apparatus 100 may generate, by combining the sub-auscultation sound data corresponding to the bioacoustic sound and the sub-auscultation sound data corresponding to the noise, training auscultation sound data for training the artificial intelligence model which outputs enhanced auscultation sound data based on the auscultation sound data.

As an example, referring to FIG. 7D, the electronic apparatus 100 may generate first training auscultation sound data 750 by mixing the second sub-auscultation sound data 720 and the third sub-auscultation sound data 730 with the fourth sub-auscultation sound data 740.

As another example, referring to FIG. 7E, the electronic apparatus 100 may generate second training auscultation sound data 760 by mixing the first sub-auscultation sound data 710 and the third sub-auscultation sound data 730 with the fourth sub-auscultation sound data 740.

At this point, the first training auscultation sound data 750 and the second training auscultation sound data 760 may correspond to the second auscultation sound data 365 of FIG. 3, and the fourth sub-auscultation sound data 740 may correspond to the bioacoustics data 370 of FIG. 3. However, it is merely an example.

As such, the electronic apparatus 100 may generate the training auscultation sound data by using the trained artificial intelligence model. Through this, a training auscultation sound data set in which corresponding correct answer data is present may be further conveniently and easily generated.

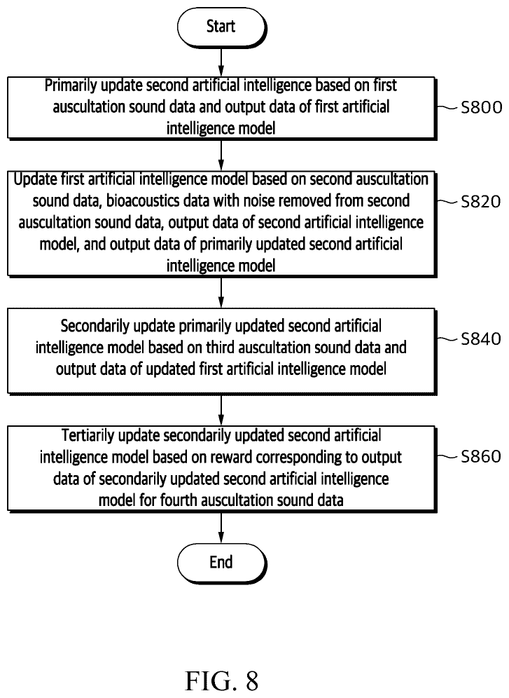

FIG. 8 is a flowchart illustrating an artificial intelligence model training method of an electronic apparatus according to an example embodiment. The above description may be applied to redundant descriptions.

In operation S800, the electronic apparatus may primarily update a second artificial intelligence based on first auscultation sound data and output data of a first artificial intelligence model.

According to an example embodiment, when primarily updating the second artificial intelligence model, the electronic apparatus may acquire first enhanced auscultation sound data by inputting the first auscultation sound data to the first artificial intelligence model, acquire second enhanced auscultation sound data by inputting the first auscultation sound data to the second artificial intelligence, acquire a first loss between the first enhanced auscultation sound data and the second enhanced auscultation sound data, and update the second artificial intelligence model so that the first loss is minimized.

According to an example embodiment, the first artificial intelligence model may include an artificial intelligence model trained in advance based on fifth auscultation sound data and bioacoustic data with noise removed from the fifth auscultation sound data.

In operation S820, the electronic apparatus may update the first artificial intelligence model based on second auscultation sound data, bioacoustics data with noise removed from the second auscultation sound data, output data of the second artificial intelligence model, and output data of the primarily updated second artificial intelligence model.

According to an example embodiment, when updating the first artificial intelligence model, the electronic apparatus may acquire third enhanced auscultation sound data by inputting the second auscultation sound data to the first artificial intelligence model, acquire fourth enhanced auscultation sound data by inputting the second auscultation sound data to the second artificial intelligence model, acquire fifth enhanced auscultation sound data by inputting the second auscultation sound data to the primarily updated second artificial intelligence model, acquire a second loss between the third enhanced auscultation sound data and the bioacoustics data, a third loss between the fourth enhanced auscultation sound data and the bioacoustics data, and a fourth loss between the fifth enhanced auscultation sound data and the bioacoustics data, and update the first artificial intelligence model so that a sum of the second loss, the third loss, and the fourth loss is minimized.

According to an example embodiment, a ratio between a first auscultation sound data set including the first auscultation sound data and a second auscultation sound data set including the second auscultation sound data is determined to be a set value.

In operation S840, the electronic apparatus may secondarily update the primarily updated second artificial intelligence model based on the third auscultation sound data and output data of the updated first artificial intelligence model.

According to an example embodiment, when secondarily updating the primarily updated second artificial intelligence model, the electronic apparatus may acquire sixth enhanced auscultation sound data by inputting third auscultation sound data to the updated first artificial intelligence model, acquire seventh enhanced auscultation sound data by inputting the third auscultation sound data to the primarily updated second artificial intelligence model, acquire a fifth loss between the sixth enhanced auscultation sound data and the seventh enhanced auscultation sound data, and update the primarily updated second artificial intelligence model so that the fifth loss is minimized.

In operation S860, the electronic apparatus may tertiarily update the secondarily updated second artificial intelligence model based on a reward corresponding to output data of the secondarily updated second artificial intelligence model for the fourth auscultation sound data.

According to an example embodiment, when tertiarily updating the secondarily updated second artificial intelligence model, the electronic apparatus may acquire eighth enhanced auscultation sound data by inputting fourth auscultation sound data to the secondarily updated second artificial intelligence model, acquire the reward which corresponds to the eighth enhanced auscultation sound data by inputting the fourth auscultation sound data and the eighth enhanced auscultation sound data to a reward model, and update the secondarily updated second artificial intelligence model so that the reward is maximized.

According to an example embodiment, the reward model may include an artificial intelligence model supervised-learned based on sixth auscultation sound data, ninth enhanced auscultation sound data acquired by inputting the sixth auscultation sound data to the secondarily updated second artificial intelligence model, and score data corresponding to the ninth enhanced auscultation sound data.

According to an example embodiment, the score data may include at least one of score data determined by a medical specialist in association with the ninth enhanced auscultation sound data and score data acquired by inputting the ninth enhanced auscultation sound data to a perception-based loss function.

According to an example embodiment, when tertiarily updating the secondarily updated second artificial intelligence model, the electronic apparatus may update the second artificial intelligence model so that a similarity between a first feature vector corresponding to the secondarily updated second artificial intelligence model and a second feature vector corresponding to the second artificial intelligence model to be tertiarily updated is present within a set range.

According to an example embodiment, the electronic apparatus may acquire first sub-auscultation sound data corresponding to a bioacoustic sound and second sub-auscultation sound data corresponding to the noise by inputting seventh auscultation sound data to a third artificial intelligence model and generate the second auscultation sound data by combining the first sub-auscultation sound data and the second sub-auscultation sound data. At this point, the bioacoustic data may include the first sub-auscultation sound data.

According to an example embodiment, the electronic apparatus may receive eighth auscultation sound data from an external electronic apparatus, acquire tenth enhanced auscultation sound data by inputting the eighth auscultation sound data to the second artificial intelligence model, and transmit the tenth enhanced auscultation sound data to the external electronic apparatus.

According to an example embodiment, the electronic apparatus may include a sound collection part and a display. The electronic apparatus may acquire eleventh enhanced auscultation sound data by inputting ninth auscultation sound data acquired through the sound collection part to the second artificial intelligence model, acquire an abnormality analysis result corresponding to the eleventh enhanced auscultation sound data by inputting the eleventh enhanced auscultation sound data to a fourth artificial intelligence model, and provide the abnormality analysis result through the display.

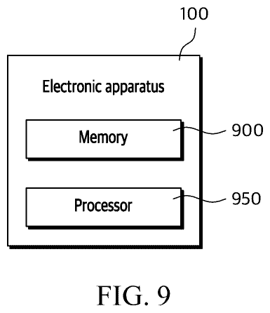

FIG. 9 is a block diagram illustrating an electronic apparatus 100 according to an example embodiment.

According to an example embodiment, the electronic apparatus 100 may include a memory 900 and a processor 950. With respect to the electronic apparatus 100 which is illustrated in FIG. 9, only elements associated with the present example embodiment are illustrated. Thus, those skilled in the art associated with the present example embodiment may understand that other elements in general use in addition to the elements illustrated in FIG. 9 may be further included.

As an example, the electronic apparatus 100 may include a transceiver (not illustrated) according to an example embodiment. The transceiver may be an apparatus for performing wired/wireless communication and communicate with an external electronic apparatus. The external electronic apparatus may be a terminal or a server. Also, a communication technology used by the transceiver may include Global System for Mobile communication (GSM), Code Division Multi Access (CDMA), Long Term Evolution (LTE), 5th Generation, (5G), a wireless local area network (WLAN), Wireless-Fidelity (Wi-Fi), Bluetooth, radio frequency identification (RFID), infrared data association (IrDA), ZigBee, near field communication (NFC), or the like.

As another example, the electronic apparatus 100 may include a sound collection part (not illustrated) according to an example embodiment. The sound collection part may be an element for acquiring a biometric signal of a subject, may be positioned on a side of the electronic apparatus 100, and may be disposed on a surface of the electronic apparatus 100.

As still another example, the electronic apparatus 100 may include a display (not illustrated) according to an example embodiment. The display may be an element for providing a variety of visual information to a user, may be positioned on a side of the electronic apparatus 100, and may be disposed on another surface of the electronic apparatus 100.

As still another example, the electronic apparatus 100 may include a sound output part (not illustrated) according to an example embodiment. The sound output part may be an element for outputting auscultation sound data and may be positioned on a side of the electronic apparatus 100.

The processor 950 may control overall operations of the server 100 and process data and a signal. The processor 950 may be formed of at least one hardware unit. In addition, the processor 950 may be operated by one or more software modules generated by executing program code stored in the memory 900.

The processor 900 may primarily update a second artificial intelligence based on first auscultation sound data and output data of a first artificial intelligence model, update the first artificial intelligence model based on second auscultation sound data, bioacoustics data with noise removed from the second auscultation sound data, output data of the second artificial intelligence model, and output data of the primarily updated second artificial intelligence model, secondarily update the primarily updated second artificial intelligence model based on the third auscultation sound data and output data of the updated first artificial intelligence model, and tertiarily update the secondarily updated second artificial intelligence model based on a reward corresponding to output data of the secondarily updated second artificial intelligence model for fourth auscultation sound data.

The electronic apparatus according to the above-described example embodiments may include a processor, a memory that stores and executes program data, a permanent storage such as a disk drive, a communication port for communicating with an external device, and a user interface device such as a touch panel, a key, and a button. Methods implemented by software modules or algorithms may be stored in a computer-readable recording medium as computer-readable code or program instructions executable in the processor. Here, the computer-readable recording medium may include a magnetic storage medium (e.g., a read-only memory (ROM), a random-access memory (RAM), a floppy disk, a hard disk, or the like), an optical reading medium (e.g., a CD-ROM or a digital versatile disc (DVD)), or the like. The computer-readable recording medium may be dispersed to computer systems connected by a network so that computer-readable codes may be stored and executed in a dispersed manner. The medium may be read by a computer, stored in the memory, and executed by the processor.

The present example embodiments may be represented by functional blocks and various processing steps. These functional blocks may be implemented by various numbers of hardware and/or software configurations that execute specific functions. For example, the present example embodiments may adopt integrated circuit configurations such as a memory, a processor, a logic circuit, and a look-up table that may execute various functions by control of one or more microprocessors or other control devices. Similarly to that elements may be executed by software programming or software elements, the present example embodiments may be implemented by programming or scripting languages such as C, C++, Java, and assembler language, including various algorithms implemented by combinations of data structures, processes, routines, or of other programming configurations. Functional aspects may be implemented by algorithms executed by one or more processors. In addition, the present example embodiments may adopt the related art for electronic environment setting, signal processing, and/or data processing, for example. The terms “mechanism”, “element”, “means”, and “configuration” may be widely used and are not limited to mechanical and physical components. These terms may include meaning of a series of routines of software in association with a processor.

The above-described embodiments are merely examples and other embodiments may be implemented within the scope of the following claims.

The various embodiments described above can be combined to provide further embodiments. Other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An artificial intelligence (AI) model training method of an electronic apparatus, the artificial intelligence model training method comprising:
primarily updating a second artificial intelligence based on first auscultation sound data and output data of a first artificial intelligence model;
updating the first artificial intelligence model based on second auscultation sound data, bioacoustics data with noise removed from the second auscultation sound data, output data of the second artificial intelligence model, and output data of the primarily updated second artificial intelligence model;
secondarily updating the primarily updated second artificial intelligence model based on third auscultation sound data and output data of the updated first artificial intelligence model; and
tertiarily updating the secondarily updated second artificial intelligence model based on a reward corresponding to output data of the secondarily updated second artificial intelligence model for fourth auscultation sound data.

2. The artificial intelligence model training method of claim 1, wherein the primarily updating of the second artificial intelligence model comprises:
acquiring first enhanced auscultation sound data by inputting the first auscultation sound data to the first artificial intelligence model with acquiring second enhanced auscultation sound data by inputting the first auscultation sound data to the second artificial intelligence;
acquiring a first loss between the first enhanced auscultation sound data and the second enhanced auscultation sound data; and
updating the second artificial intelligence model so that the first loss is minimized.

3. The artificial intelligence model training method of claim 1, wherein the updating of the first artificial intelligence model comprises:
acquiring third enhanced auscultation sound data by inputting the second auscultation sound data to the first artificial intelligence model with acquiring fourth enhanced auscultation sound data by inputting the second auscultation sound data to the second artificial intelligence model and acquiring fifth enhanced auscultation sound data by inputting the second auscultation sound data to the primarily updated second artificial intelligence model;
acquiring a second loss between the third enhanced auscultation sound data and the bioacoustics data, a third loss between the fourth enhanced auscultation sound data and the bioacoustics data, and a fourth loss between the fifth enhanced auscultation sound data and the bioacoustics data; and
updating the first artificial intelligence model so that a sum of the second loss, the third loss, and the fourth loss is minimized.

4. The artificial intelligence model training method of claim 1, wherein the secondarily updating of the primarily updated second artificial intelligence model comprises:
acquiring sixth enhanced auscultation sound data by inputting the third auscultation sound data to the updated first artificial intelligence model with acquiring seventh enhanced auscultation sound data by inputting the third auscultation sound data to the primarily updated second artificial intelligence model;
acquiring a fifth loss between the sixth enhanced auscultation sound data and the seventh enhanced auscultation sound data; and
updating the primarily updated second artificial intelligence model so that the fifth loss is minimized.

5. The artificial intelligence model training method of claim 1, wherein the first artificial intelligence model includes an artificial intelligence model trained in advance based on fifth auscultation sound data and bioacoustic data with noise removed from the fifth auscultation sound data.

6. The artificial intelligence model training method of claim 1, wherein the tertiarily updating of the secondarily updated second artificial intelligence model comprises:
acquiring eighth enhanced auscultation sound data by inputting the fourth auscultation sound data to the secondarily updated second artificial intelligence model;
acquiring the reward which corresponds to the eighth enhanced auscultation sound data by inputting the fourth auscultation sound data and the eighth enhanced auscultation sound data to a reward model; and
updating the secondarily updated second artificial intelligence model so that the reward is maximized.

7. The artificial intelligence model training method of claim 6, wherein the reward model includes an artificial intelligence model supervised-learned based on sixth auscultation sound data, ninth enhanced auscultation sound data acquired by inputting the sixth auscultation sound data to the secondarily updated second artificial intelligence model, and score data corresponding to the ninth enhanced auscultation sound data.

8. The artificial intelligence model training method of claim 7, wherein the score data includes at least one of:
score data determined by a medical specialist in association with the ninth enhanced auscultation sound data; and
score data acquired by inputting the ninth enhanced auscultation sound data to a perception-based loss function.

9. The artificial intelligence model training method of claim 1, wherein the tertiarily updating of the secondarily updated second artificial intelligence model comprises updating the second artificial intelligence model so that a similarity between a first feature vector corresponding to the secondarily updated second artificial intelligence model and a second feature vector corresponding to the second artificial intelligence model to be tertiarily updated is present within a set range.

10. The artificial intelligence model training method of claim 1, further comprising:
acquiring first sub-auscultation sound data corresponding to a bioacoustic sound and second sub-auscultation sound data corresponding to noise by inputting seventh auscultation sound data to a third artificial intelligence model; and
generating the second auscultation sound data by combining the first sub-auscultation sound data and the second sub-auscultation sound data,
wherein the bioacoustic data includes the first sub-auscultation sound data.

11. The artificial intelligence model training method of claim 1, wherein a ratio between a first auscultation sound data set including the first auscultation sound data and a second auscultation sound data set including the second auscultation sound data is determined to be a set value.

12. The artificial intelligence model training method of claim 1, further comprising:
receiving eighth auscultation sound data from an external electronic apparatus;
acquiring tenth enhanced auscultation sound data by inputting the eighth auscultation sound data to the second artificial intelligence model; and
transmitting the tenth enhanced auscultation sound data to the external electronic apparatus.

13. The artificial intelligence model training method of claim 1, wherein the electronic apparatus includes:
a sound collection part; and
a display, and
the artificial intelligence model training method further comprises:
acquiring eleventh enhanced auscultation sound data by inputting ninth auscultation sound data acquired through the sound collection part to the second artificial intelligence model;
acquiring an abnormality analysis result corresponding to the eleventh enhanced auscultation sound data by inputting the eleventh enhanced auscultation sound data to a fourth artificial intelligence model; and
providing the abnormality analysis result through the display.

14. A non-transitory computer-readable recording medium in which a program for executing the artificial intelligence model training method of claim 1 in a computer is recorded.

15. An electronic apparatus comprising:
a memory; and
a processor,
wherein the processor is configured to:
primarily update a second artificial intelligence based on first auscultation sound data and output data of a first artificial intelligence model;
update the first artificial intelligence model based on second auscultation sound data, bioacoustics data with noise removed from the second auscultation sound data, output data of the second artificial intelligence model, and output data of the primarily updated second artificial intelligence model;
secondarily update the primarily updated second artificial intelligence model based on third auscultation sound data and output data of the updated first artificial intelligence model; and
tertiarily update the secondarily updated second artificial intelligence model based on a reward corresponding to output data of the secondarily updated second artificial intelligence model for fourth auscultation sound data.
